(19) United States
(12) Patent Application Publication
(10) Pub. No.: US 2026/0011438 A1
(43) Pub. Date: Jan. 8, 2026

(54) MEDICAL DEVICE MANAGEMENT SYSTEM

(71) Applicant: Willow Laboratories, Inc., Irvine, CA (US)

(72) Inventor: Marcelo M. Lamego, Cupertino, CA (US)

(21) Appl. No.: 19/249,750

(22) Filed: Jun. 25, 2025

Related U.S. Application Data
(63) Continuation of application No. 18/165,675, filed on Feb. 7, 2023, now Pat. No. 12,367,973.
Application No. 18/165,675 is a division of application No. 15/067,061, filed on Mar. 10, 2016, now Pat. No. 11,596,363.
Application No. 15/067,061 is a continuation of application No. PCT/US2014/055042, filed on Sep. 10, 2014.
(60) Provisional application No. 61/877,181, filed on Sep. 12, 2013.

(51) Int. Cl. G16H 40/40; A61B 5/00; A61B 5/145; A61B 5/1455; A61B 5/1459; A61B 5/1495; G16H 10/60; G16H 40/63

(52) U.S. Cl. CPC G16H 40/40; A61B 5/0002; A61B 5/0004; A61B 5/14532; A61B 5/14546; A61B 5/14551; A61B 5/1459; A61B 5/1495; G16H 10/60; G16H 40/63; A61B 5/7282

(57) ABSTRACT

The present disclosure provides systems and methods for calibrating medical devices and processing physiological measurements using a medical device management system. As an example, the medical device can be a handheld glucometer configured for invasive testing and non-invasive testing of physiological parameters of a patient.

### RELATED APPLICATIONS

### Incorporation by Reference to any Priority Applications

Any and all applications for which a foreign or domestic priority claim is identified in the Application Data Sheet as filed with the present application are incorporated by reference under 37 CFR 1.57 and made a part of this specification.

### BACKGROUND

Medical device manufacturers are continually increasing the processing capabilities of physiological monitors that process signals based upon the attenuation of light by a tissue site. In general, such physiological monitoring systems include one or more optical sensors that irradiate a tissue site and one or more photodetectors that detect the optical radiation after attenuation by the tissue site. The sensor communicates the detected signal to a physiological monitor, which removes noise and preprocesses the signal. Advanced signal processors then perform time domain and/or frequency domain processing to determine blood constituents and other physiological parameters.

Manufacturers have advanced basic pulse oximeters from devices that determine measurements for blood oxygen saturation (Sp02), pulse rate (PR) and plethysmographic information to read-through-motion oximeters and to cooximeters that determine measurements of many constituents of circulating blood. For example, Masimo Corporation of Irvine Calif. (“Masimo”) manufactures pulse oximetry systems including Masimo SET® low noise optical sensors and read through motion pulse oximetry monitors for measuring Sp02, PR and perfusion index (PI). Masimo optical sensors include any of Masimo LNOp®, LNCS®, SofTouch™ and Blue™ adhesive or reusable sensors. Masimo pulse oximetry monitors include any of Masimo Rad-8®, Rad-50, Rad®-5v or SatShare® monitors. Such advanced pulse oximeters and low noise sensors have gained rapid acceptance in a wide variety of medical applications, including surgical wards, intensive care and neonatal units, general wards, home care, physical training, and virtually all types of monitoring scenarios.

Many innovations improving the measurement of blood constituents are described in at least U.S. Pat. Nos. 6,770,028; 6,658,276; 6,157,850; 6,002,952; 5,769,785 and 5,758,644, which are assigned to Masimo and are incorporated by reference herein. Corresponding low noise optical sensors are disclosed in at least U.S. Pat. Nos. 6,985,764; 6,088,607; 5,782,757 and 5,638,818, assigned to Masimo and hereby incorporated in their entirety by reference herein.

Advanced blood parameter measurement systems include Masimo Rainbow® SET, which provides measurements in addition to Sp02, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Advanced parameter measurement systems mayaiso include acoustic monitoring such as acoustic respiration rate (RRa™) using a Rainbow Acoustic Sensor™ and Rad-87™ monitor, available from Masimo. Such advanced pulse oximeters, low noise sensors and advanced parameter systems have gained rapid acceptance in a wide variety of medical applications, including surgical wards, intensive care and neonatal units, general wards, ho me care, physical training, and virtually all types of monitoring scenarios. An advanced parameter measurement system that includes acoustic monitoring is described in U.S. Pat. Pub. No. 2010/0274099, filed Dec. 21, 2009, titled Acoustic Sensor Assembly, assigned to Masimo and incorporated in its entirety by reference herein.

Innovations relating to other advanced blood parameter measurement systems are described in at least U.S. Pat. No. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, CA (Cercacor) and all incorporated in their entirety by reference herein.

### SUMMARY

In some embodiments the present disclosure provides a method for processing physiological measurements. The method including receiving, by a medical device management system, physiological measurement data from a portable medical device. The physiological measurement data includes identification information associated with a user of the portable medical device. The method also includes identifying a user account of the medical device management system based on identification data, processing the physiological measurement data to determine at least one physiological parameter associated with the physiological measurement data, transmitting the determined at least one physiological parameter to the portable medical device for display, and storing the physiological measurement data and the determined at least one physiological parameter in the identified user account.

In some embodiments the present disclosure provides a method for calibrating a portable medical device. The method includes receiving a request for calibration from a portable medical device. The request includes identification information associated with the medical device. The method further includes identifying an account associated with the medical device based on the identification information, determining that the device needs calibration based on the information stored in the account, sending a signal to the medical device to initiate a calibration mode on the medical device, receiving calibration data from the medical device, processing the calibration data to determine a calibration of the device, transmitting an updated calibration to medical device, and storing the updated calibration in the account associated with the medical device.

In some embodiments the present disclosure provides a medical device management system including a data store and a computing device. The data store can be configured to store user account information associated with a plurality of user accounts. The computing device is in communication with the data store and can be configured to receive a measurement request from a portable medical device. The measurement request can include physiological measurement data. The computing device can be further configured to identify a user account of the plurality of user accounts associated with the measurement request, process the physiological measurement data to determine at least one physiological parameter associated with the physiological measurement data, transmit the determined at least one physiological parameter to the portable medical device for display and storing the physiological measurement data and the determined at least one physiological parameter in the identified user account.

For purposes of summarizing the invention, certain aspects, advantages and novel features of the invention have been described herein. Of course, it is to be understood that not necessarily all such aspects, advantages or features will be embodied in any particular embodiment of the invention.

### BRIEF DESCRIPTION OF THE DRAWINGS

The foregoing aspects and many of the attendant advantages will become more readily appreciated as the same become better understood by reference to the following detailed description, when taken in conjunction with the accompanying drawings:

FIG. 1 illustrates a block diagram depicting an illustrative embodiment of an operating environment for a medical device management system.

FIG. 2 illustrates a perspective view of an embodiment of a handheld glucometer.

FIG. 3 illustrates an end view of the embodiment of the handheld glucometer from FIG. 2.

FIG. 4 illustrates an opposite end view of the embodiment of the handheld glucometer from FIG. 2.

FIG. 5 illustrates an exploded view of the embodiment of the handheld glucometer from FIG. 2.

FIG. 6 illustrates a method of updating the calibration of a medical device using a medical device management system.

### DETAILED DESCRIPTION

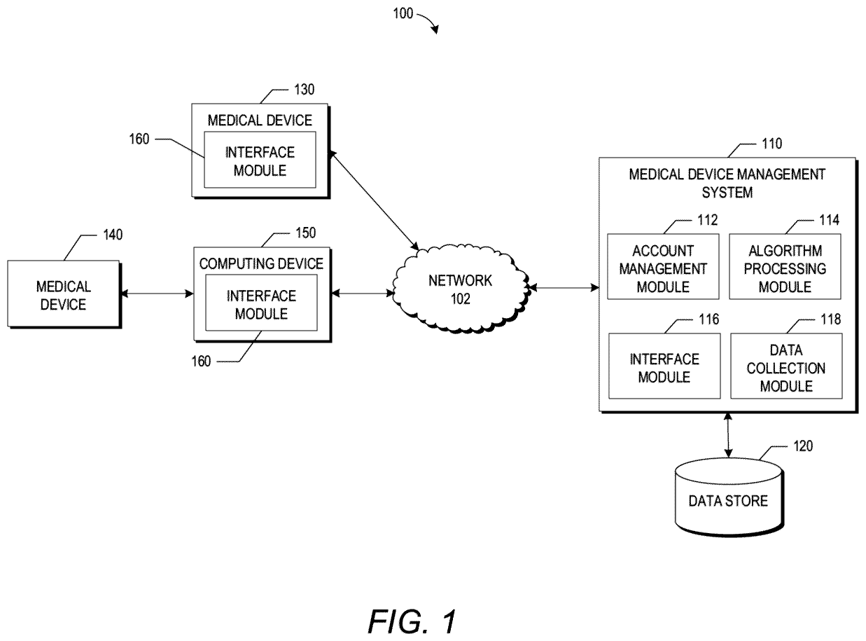

FIG. 1 illustrates an embodiment of an operating environment 100 for a medical device management system 110. The operating environment includes a medical device management system 110 configured to communicate with a plurality of medical devices 130 and 140 over a network 102. The medical device management system 110 can also be referred to as a network-based or cloud-based management system.

Those skilled in the art will appreciate that the communication network 102 may be any wired network, wireless network or combination thereof. In addition, the communication network 102 may be a personal area network, local area network, wide area network, cable network, satellite network, cellular telephone network, or combination thereof. Protocols and components for communicating via the Internet or any of the other aforementioned types of communication networks are well known to those skilled in the art of computer communications and thus, need not be described in more detail herein.

In this embodiment, the medical device management system 110 includes an account management module 112, an algorithm processing module 114, an interface module 116, and a data collection module 118. The medical device management system 110 is in communication with a data store 120. The data store 120 can store data received from the medical devices 130 and 140.

In general, the word module, as used herein, refers to logic embodied in hardware or firmware, or to a collection of software instructions stored on a non-transitory, tangible computer-readable medium, possibly having entry and exit points, written in a programming language, such as, for example, C, C++, C#, or Java. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules may be stored in any type of computer-readable medium, such as a memory device (e.g., random access, flash memory, and the like), an optical medium (e.g., a CD, DVD, BluRay, and the like), firmware (e.g., an EPROM), or any other storage medium. The software modules may be configured for execution by one or more CPUs in order to cause the medical device management system 110 to perform particular operations.

It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware. Generally, the modules described herein refer to logical modules that may be combined with other modules or divided into sub-modules despite their physical organization or storage.

The medical devices 130 and 140 can be configured to measure and record physiological signals from a user. The physiological signals including, but not limited to, blood pressure (diastolic), blood pressure (systolic), PR, glucose, total hemoglobin (SpHb), SpO2, PI, venous oxygen saturation (SpVO2), pleth variability index (PVI), SpCHOL, SpBUN, SpHDL, and/or other physiological parameters.

The medical devices 130 and 140 can have an associated sensor, such as an optical sensor, to monitor physiological parameters. One example of an optical sensor is described in detail with respect to U.S. patent application Ser. No. 13/646,659 titled Noninvasive Blood Analysis System, filed Oct. 5, 2012, assigned to Cercacor and incorporated in its entirety by reference herein. A blood glucose monitor is described in detail with respect to U.S. patent application Ser. No. 13/308,461 titled Handheld Processing Device Including Medical Applications for Minimally and Noninvasive Glucose Measurements, filed Nov. 30, 2011, assigned to Cercacor and incorporated in its entirety by reference herein. A blood glucose monitor and sensor are described in detail with respect to U.S. patent application Ser. No. 13/473,477 titled Personal Health Device, filed May 16, 2012, assigned to Cercacor and incorporated in its entirety by reference herein. A blood glucose calibration system is described in detail with respect to U.S. patent application Ser. No. 13/726,539 titled Blood Glucose Calibration System, filed Dec. 24, 2012, assigned to Cercacor and incorporated in its entirety by reference herein.

The medical devices 130 and 140 can communicate with the medical device management system 110 via the network 102. In some embodiments, a medical device can be a network-capable device, such as medical device 130. The medical device 130 can be configured to communicate directly with the medical device management system 110. The medical device 130 can have an interface module 160 configured to manage communication between the medical device management system 110 and the medical device 130. The interface module 160 can be specific to the medical device 130 and each type of medical device can have a different interface module 160. Some medical devices, such as medical device 140, communicate with the medical device management system 110 via a host computing device 150.

The computing device 150 can correspond to a wide variety of devices or components that are capable of initiating, receiving or facilitating communications over the communication network 102 including, but not limited to, personal computing devices, hand held computing devices, integrated components for inclusion in computing devices, smart phones, modems, personal digital assistants, laptop computers, media devices, and the like.

The computing device 150 can have an interface module 160 that can be configured to interface with the medical device management system 110. The interface module 160 can be configured to provide a user with access to the medical device management system 110. The interface module 160 can be an application that operates on the computing device 150. The application can be configured to recognize the medical device 140 when the device is in communication with the computing device 150. The interface module 160 can be used when the medical device 140 is not capable of communicating directly with the medical device management system 110 over the network. The medical device 140 can communicate with the interface module 160 via a physical connection to a computing device 150 (e.g., a USB connection) or using wireless communication protocols (e.g., WiFi, Bluetooth, etc.).

The interface module 160 can be configured to communicate with the medical device management system and provide and receive information from the medical device. The information provided by the medical device to the medical device management system 110 can include information such as device serial number, calibration information, synchronization information, biometric data, and other types of data or information. For simplicity, reference will generally be made to the medical device 130 when describing interactions between a medical device and the medical device management system 110. The functionality described with relation to medical device 130 can be implemented on medical device 140, either directly or through another computing device. Depending on the specific type of medical device, none, some or all of the features and functionality discussed herein may be implemented locally on the medical device.

The medical device management system 110 can include a user portal and a back end infrastructure. The medical device management system 110 can store, organize and present medical data collected by the medical device to the user. The account management module 112 can manage the user accounts in the medical device management system 110. The algorithm processing module 114 can be configured to utilize algorithms to dynamically and quickly determine blood constituent values that would not be calculable locally on a portable medical device. The interface module 116 can be configured to interface with the medical devices 130 and 140 via the respective device-side interface modules 160. The interface module 116 can also be configured to manage a web-based interface. Further, the interface module 116 can be responsible for synchronization of the medical with the medical device management system 110. The interface module 116 can also be responsible for uploading data to the medical device management system 110. The data collection module 118 can store, organize and present medical data collected by the devices to the users and interface with the data store 120.

The medical device management system 110 can utilize an account-based management system. Each user can set up a user account that can be associated with one or more medical devices. The user can provide information during an account registration process, such as demographic information, age, gender, date of birth, height, weight, credit card information, and/or other user information.

Users can interact with the medical device management system 110 to initialize, configure, synchronize and manage their medical devices. The medical device management system 110 can perform data processing of the medical data collected from the medical devices. When a user has registered their medical device(s) to their user account on the medical device management system 110, the user can have access to medical data collected by their medical device. Each user can use the medical device management system 110 as a portal through which they can monitor and manage their medical data over time. The medical device management system 110 can sort through the user's data and present it in relevant views (such as monthly trends, yearly trends, after meal trends, etc.). In some embodiments, the user can personalize the medical device, such as creating a name for the device, selecting the language of the device, privacy settings, alarms, thresholds, and other options. The privacy settings can allow the user to choose whether to share their data with others. For example, the user can choose not to share the data, or the user can share the data with others, such as with their family, their doctor, and/or everyone.

Prior to use, the interface module 160 communicate with the medical device management system to initialize and configure the medical device 130 and synchronize the medical device 130 with a user's personal account within the medical device management system 110. After configuration, the medical device can be registered and synced to the user's medical device management system account and data collected on medical device can be synchronized and uploaded to the user's medical device management system account. In some embodiments, a user can access the medical device and make configuration settings for the medical device via a web interface by logging in to the medical device management system. Changes made to the configuration settings in the web interface can be pushed to the medical device during a synchronization procedure. For example, if there is a change detected, the interface module 160 can retrieve the configuration table from medical device management system 110 and apply the configuration to the connected device. Identification information associated with a user account and/or user device can be used to associate the physiological measurement data received from a device with a user account. The identification information can include information such as a user ID, device ID (e.g., serial number), user credentials, tokens, or other types of information that can be used by the medical device management system. For example, when the medical device management system receives physiological data, the data could include identification information that can be used to identify the user account and/or device associated with the physiological measurement data.

In one embodiment of the initialization process, the interface module 160 can retrieve identification information or data such as, user credentials, device ID (e.g., serial number), and/or other required and/or relevant information from the device. The information can be sent to the medical device management system 110. The receipt of information by the medical device management system 110 from the medical device 130 can trigger the medical device management system 110 to provide an authentication token to the medical device 130. The authentication token can be provided to the medical device 130 via the interface module 160, which can be stored on the device for authentication and identification purposes.

The medical device management system 110 can be configured to allow multiple users to use the same medical device. One user can be designated as the device administrator within the medical device management system 110. After the medical device is registered and initialized for use by a device administrator, additional users can be associated with the medical device. The additional users can use the medical device and have their personal medical data managed on a separate user account with the medical device management system 110. In some embodiments, an administrator can grant additional users access by sending an invitation via their medical device management system account. An unregistered user (e.g., guest user) may still be able to use the medical device, however, the medical device management system may not store and track the unregistered user's data.

A medical device that has been registered can synchronize the data stored on the medical device with the medical device management system 110. The interface module 160 can retrieve the physiological data from the medical device and send it to the medical device management system 110. The medical device management system can store the physiological data in the data store 120. The stored data can be retrieved and sorted by the user.

Data sent from the medical device to the medical device management system 110 can trigger creation of a file with information associated with the physiological data. In one embodiment the file can include, a timestamp, a location of a binary file (raw physiological data), and location that an output file should be stored.

### Online Data Processing

The medical device management system 110 can provide massive computing capability in the cloud for many users and devices. The increased computing power can be used to run increasingly more complex algorithms as well as supporting multiple devices simultaneously. The algorithms running on the medical device management system 110 can be updated. The version and specifics of the algorithm can also be traceable and there can be a log of each update.

When the medical device management system 110 receives new physiological data from a medical device, it can store the data in the data store 120. The medical device management system 110 can create a reference file, such as an XML file, referencing the new physiological data and invoking the algorithm processing module 114. The reference file can provide the information necessary to access the physiological information, run the required algorithm, calculate the results (or return an error code if unable to generate a result), and then create the desired output file. The output file can include information such as measured parameters, processing time, errors that may have occurred, algorithm version, and timestamp. The medical device management system can send the results back to the device to be displayed for the user. The medical device management system can save the output results in the database for future use.

Since the medical device management system 110 can have a virtually unlimited storage capacity, more complex algorithms can be implemented which utilize a patient's historical data in order to improve future measurements. For example, a patient-unique calibration may utilize historical measurements and calibration points to reach a better accuracy. The medical device management system can store and retrieve data and filter results by device ID, User ID, date etc., so that the algorithm knows which files are relevant for each particular subject and should be used in generating a calibration.

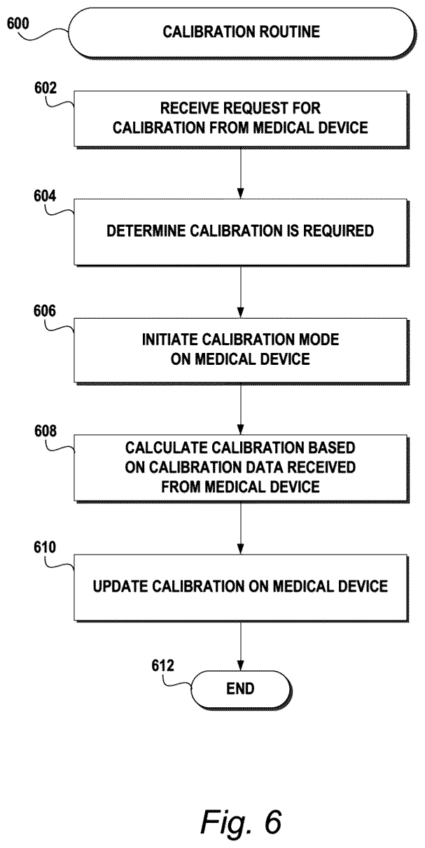

The medical device 130 can determine whether the medical device management system 110 is ready to begin processing data. If medical device management system is not ready, the medical device can return an error after a timeout period. If the medical device management system 110 is ready, the system can begin caching resources in preparation for data processing. The device can begin collecting and streaming physiological data associated with the user to the medical device management system 110. The medical device management system 110 can process the collected data and determine one or more physiological measurements based on the physiological data. The medical device can be configured to provide the raw data to the medical device management system 110 for processing. The raw data may undergo some processing (e.g., filtering) prior to being transferred to the medical device management system. In some embodiments, the medical device 130 can be configured to determine whether there is a connection to the medical device management system 110 prior to processing the data. For example, if there is no connection, the device may process the physiological data locally. Whereas, if there is a connect to the medical device management system 110, the device can send the data to the system 110 for processing. The system can also calibrate the medical device, which will be further discussed with relation to FIG. 6. Handheld Glucometer

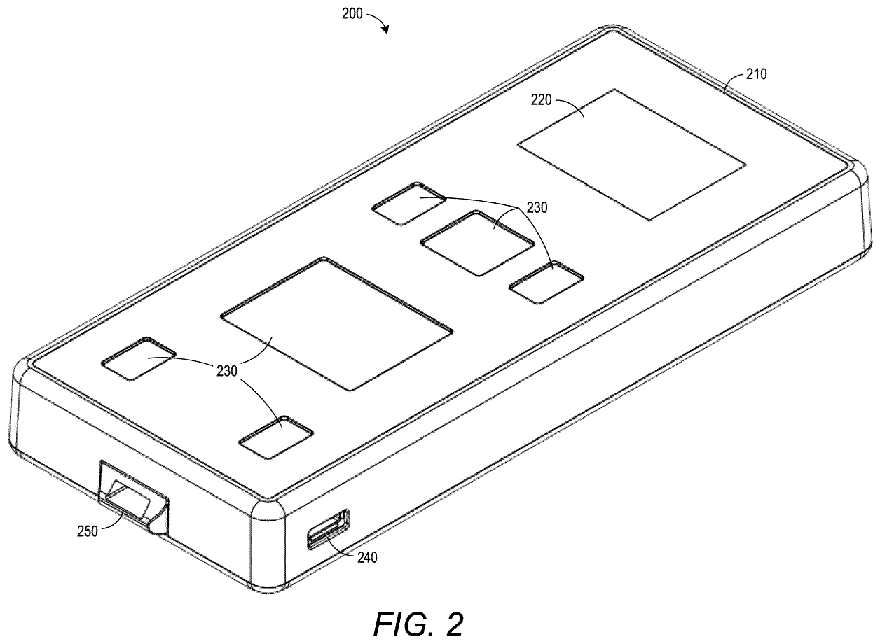

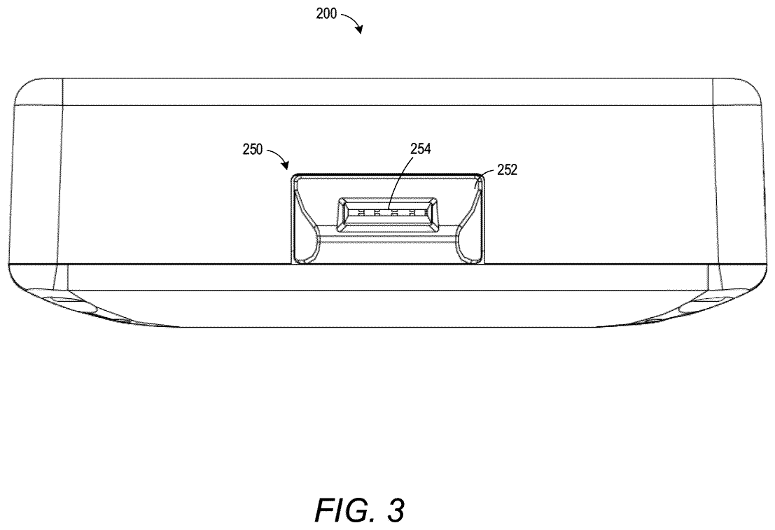

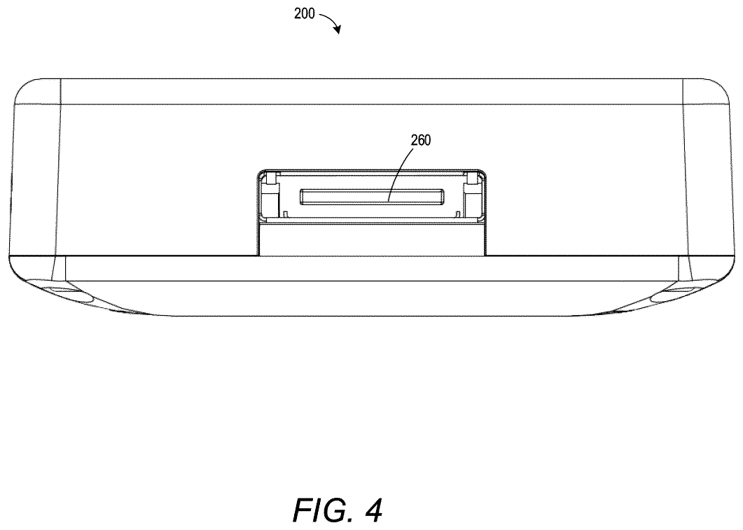

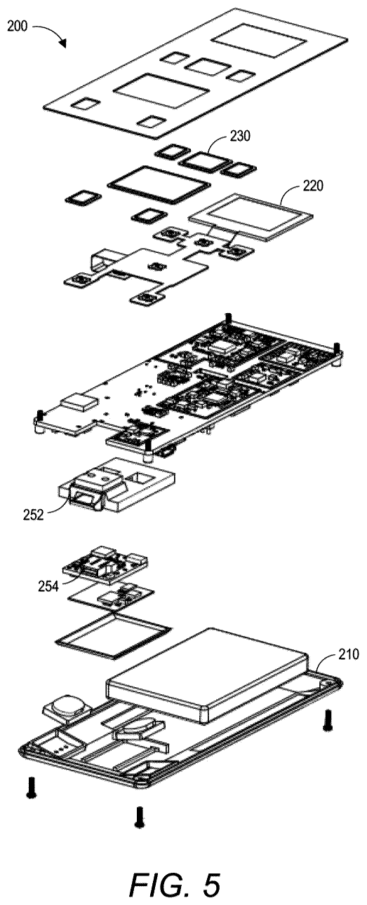

With reference now to FIGS. 2-5, an illustrative embodiment of a handheld medical device 200 is illustrated. The handheld medical device 200 can also be referred to as a handheld glucometer. The handheld glucometer 200 includes a housing 210, a display 220, a plurality of control buttons 230, an I/O port 240, a glucose reader 250, and a sensor connector 260.

The handheld glucometer 200 can be utilized for invasive and/or non-invasive blood glucose monitoring or non-invasive partial blood panel monitoring by home users in a non-clinical setting or trained individuals in a clinical setting. The handheld glucometer 200 can perform one or more of the following physiological measurements: invasive glucose testing, non-invasive blood glucose testing, Oxygen Saturation (SpO2), Total Hemoglobin (SpHb), Alkaline Phosphatase (SpALP), Total Cholesterol (SpChol), High-Density Lipoprotein (SpHDL), Total Cholesterol Divided by High Density Lipoprotein (SpChol/SpHDL).

The handheld glucometer 200 can perform invasive blood glucose measurements when the user lancets their finger for a capillary blood sample and places it onto a glucose test strip that is inserted into the handheld glucometer 200. The invasive blood glucose measurements can be used for at least two functions. First, the invasive measurements can be used by the handheld glucometer 200 during calibration. The handheld glucometer 200 can request that invasive calibration measurements be taken from time to time, such as during the initial use of the device in order to set a standard of calibration for the patient and the non-invasive sensor. Second, the user can take invasive measurements to test the accuracy of the non-invasive blood glucose measurements or when the user prefers to have an invasive measurement taken. The handheld glucometer 200 can help to reduce the frequency of pain associated with typical home blood glucose meters that require invasive blood draws approximately 4-7 times per day. Advantageously, with the handheld glucometer 200 a user may be able to reduce the number of invasive blood draws to 1-2 per week if they can be replaced with 4-7 non-invasive measurements per day, which can reduce the pain associated with frequent lancing and reduce the likelihood of tissue damage.

The handheld glucometer 200 can be used in conjunction with the medical device management system 110. The handheld glucometer 200 can communicate with the medical device management system 110 via a computing device 150. In some embodiments, the handheld glucometer 200 can be physically connected to the computing device 150, such as a USB connection, or a wireless connection, such as a Bluetooth connection. The computing device can be a mobile computing device such as a smart phone, or another computing device such as a desktop computer. The medical device management system 110 can allow the user to review trends and user logged variables that can contribute to highs and lows in their glucose values. The results can be shared with family, friends, and care givers.

The display 220 can display text and graphics. In one embodiment, the display 220 can be an OLED display. In one embodiment, the display 220 can have a resolution of 128×96 pixels. The display can have a viewing angle that is greater than or equal to about 45 degrees on all axes. The display 220 can have a user interface. The user interface can be configured to show a wireless connectivity state, such as a Bluetooth connection state. The user interface can be configured to show battery capacity icon with states of charge remaining and/or charge state. The user interface can display a real-time clock that can be accurate to within 10 minutes per year, which the handheld glucometer 200 can use to time stamp each test. The handheld glucometer 200 can provide the user with the option to select from available regional time zones. The user interface can track and display an average on a preconfigured number of days and can display a total number of glucose results. In some embodiments, the handheld glucometer 200 can prevent users from omitting non-invasive or invasive glucose test results.

The handheld glucometer 200 can have various operating states, such as an off mode where the processor is powered off; a low-power operating mode where the processor power is minimized, the display 220 is off, and wireless connections can be maintained; and a normal operating mode where the display 220 is on and the processor is fully operating. The handheld glucometer 200 can transition from low power mode to normal operating mode by a user press of a hard button, a user inserting an invasive strip, or a user attaching a non-invasive sensor.

The handheld glucometer 200 can have various test configurations, such as on-demand where the handheld glucometer 200 ready for an invasive glucose test when a user inserts an invasive strip in the device, or ready for a non-invasive glucose test when a user connects the non-invasive sensor. The handheld glucometer 200 can perform non-invasive measurements in less than 180 seconds (e.g., three 60 second measurements if/when necessary). The handheld glucometer 200 can perform invasive measurements in less than 10 seconds.

The handheld glucometer 200 can have a plurality of control buttons, including a “Start/Stop” button, “Plus/Minus” buttons, a “Trend Button” configured to show previous tests, a volume button configured to adjust the volume, and a wireless connectivity button. The handheld glucometer 200 can have a Micro USB connector to facilitate battery charging, data transfer of measurement data, and software upgrades.

The handheld glucometer 200 can have a glucose reader 250 comprising a shell 252 and a slot 254 for the invasive strip glucose reader. The glucose strip reader 250 can be on different side of device from sensor connector 260. The glucose strip reader 250 can facilitate invasive testing and calibration testing. The slot 254 can be illuminated to facilitate the insertion of the test strip into the slot by the user.

The handheld glucometer 200 can automatically recognize when a test strip is inserted into the slot 254. The handheld glucometer 200 can instruct a user on how to operate the device during a testing procedure. The display 220 can show a percentage complete or seconds remaining count-down during an invasive glucose measurement. The display 220 can distinguish between invasive glucose values and other measured parameters.

For non-invasive measurements, the display 220 can show a percentage complete or count-down during a measurement. The handheld glucometer 200 can have a non-invasive glucose calibration capability that has a frequency that can be automatically controlled by device and/or manually run by a user at any time. The handheld glucometer 200 can have a lock out mechanism to prevent non-invasive glucose tests if the system has not been successfully calibrated. The display 220 can distinguish between non-invasive glucose values and other measured parameters on device.

The handheld glucometer 200 can have a quality control test mode to help verify that the system is operating within specifications. In the quality control test mode, the handheld glucometer 200 tests the measurement values associated with control test strips that utilize control solutions. The control test strips have known associated test values. The handheld glucometer 200 can also have a non-invasive quality control method that permits a user or manufacturing personnel to check the validity of the non-invasive system with a rainbow parameter sensor.

The handheld glucometer 200 can have a radio interface to communicate via radio communication, such as Bluetooth 2.0-4.0 and Bluetooth low energy (BLE), in order to facilitate data transfer (measurements and physiological data), connection to a computing device, such as a mobile device. The handheld glucometer 200 has non-volatile memory, such as a non-removable MicroSD card, to maintain system software and user measurements.

The handheld glucometer 200 can have a rechargeable battery, such as a 1600mAH Li+ battery. The handheld glucometer 220 can perform a minimum of 20 consecutive non-invasive test measurements from full charge. The handheld glucometer 200 can perform measurements while charging and charge at full rate when in low power mode. At full rate, the handheld glucometer 200 can charge the battery in less than 6 hours. The handheld glucometer 200 can operate in a standby mode for a minimum of 18 hours per full charge.

The sensor interface 260 can connect to a non-invasive sensor such as an optical sensor, such as one of the sensors described above that be connected to the medical devices 130 and 140. Some measurement values generated by a non-invasive sensor can include oxygen saturation (SpO2), pulse rate (PR), perfusion index (PI), total hemoglobin (SpHb), alkaline phosphatase (SpALP), total cholesterol (SpChol) (3406), high-density lipoprotein (SpHDL), total cholesterol divided by high density lipoprotein (SpChol/SpHDL), and non-invasive glucose (SpGlu). Other parameters can be viewable through the host device or a web application.

The handheld glucometer 200 can be user configurable by a host device (e.g., a computing device in communication with the handheld glucometer) or a website to make changes to the user interface. The user can determine a priority for non-invasive measurement and determine the display characteristics of the measurement values. The user can determine which measurement value is the default measurement after a non-invasive test is performed. The handheld glucometer 200 can have user set parameters for upper and lower limit notification control settings. The parameters can be set from the host device or a web-based interface with the medical device management system 110. The user can set restrictions on the access and notification of the previous test results stored in the medical device management system. The handheld glucometer 200 can have measurement alerts that can have a visual and/or an audio alert to notify the user when measurement levels, invasive or non-invasive, exceed a specified measurement range. The medical device management system can have a diabetes management system to display historical data and trends.

With reference now to FIG. 6, a calibration routine 600 for a medical device is illustrated. The calibration routine 200 can be performed generally by the medical device management system, and more specifically, by one or more modules of the medical device management system 110, such as the algorithm processing module 114, the interface module 116, and/or the data collection module 118. The calibration routine can be for single point calibration, multi-point calibration and/or other medical device specific calibration techniques.

At block 602, the medical device management system 110 can receive a request for calibration from the medical device 202. The medical device 130 can query the medical device management system 110 to determine whether calibration is required. The medical device 130 can query the medical device management system 110 each time measurement data is collected, after a defined number of measurements, after a determined time period, or other criteria.

At block 604, the medical device management system 110 can determine calibration of the medical device is required. The medical device management system 110 can determine that calibration is required based on the information contained in the request received from the medical device 130. In some embodiments, the medical device management system 110 determines if and when calibration is required based on previously stored data. In such cases, the medical device management system 110 may actively initiate calibration of the medical device 130 without previously receiving a request from the medical device 130. If the medical device management system 110 determines that no calibration required, data acquisition can occur as normal.

If calibration is required, at block 606, the calibration mode can be initiated on the medical device. The calibration mode can be a separate function on the device that handles communication with the medical device management system 110 independently of the data collection. The calibration mode on the medical device can be configured to lock the medical device from further use until calibration is completed. In calibration mode the medical device can send calibration data, such as measurement values, error codes, timestamps, device ID, sensor ID, and other information to the medical device management system 110. The specific calibration information required by the medical device management system 110 is dependent on the type of the device, type of calibration, and/or other device or system specific information.

In one embodiment for a non-invasive glucose calibration, the device can perform a manual entry calibration and a calibration using a built in strip reader. For manual entry calibration, the user can have the option of manually entering their glucose value into the medical device. Manual entry gives the flexibility to use a reference device other than the internal strip reader to help with calibration. For a strip reader calibration, the user can immediately measure their glucose value with a built in strip reader based on an invasive test. The device can also measure the glucose value using a non-invasive test and the values can be sent to the medical device management system 110 to calculate the results of the calibration.

At block 608, the medical device management system 110 calculates a calibration for the medical device based on the calibration data received from the medical device 130. The medical device management system 110 can determine the calibration based on the specific calibration information stored in the system. For example, a lookup table may be used to determine the calibration for the device. More advanced algorithms and patient specific calibrations may be used based on the information provided by the medical device.

The medical device management system 110 can store all of the data associated with a specific patient and a specific device, thereby allowing the system to tailor the calibration to the specific patient and the specific device. For example, a patient-unique calibration may utilize historical measurements and calibration points to reach a better accuracy. The medical device management system can store and retrieve data and filter results by device ID, User ID, date etc., so that the algorithm knows which files are relevant for each particular subject and should be used in generating the patient unique calibration. The calibration information can be stored in the data store 120, so that it can be referenced at a later time.

After the calculation of the calibration is complete, at block 610, the calibration can be updated on the medical device. The medical device management system 110 can provide the updated calibration data to the medical device 130. After updating the calibration, the medical device can transition from the calibration mode to measurement mode, which can allow the user to perform measurements as required. The calibration routine ends at block 612.

In addition to those processes described above, other processes and combination of processes will be apparent to those of skill in the art from the present disclosure. Those of skill will further appreciate that the various illustrative logical blocks, modules, and steps described in connection with the embodiments disclosed herein may be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of the present invention.

The various illustrative logical blocks, modules, and steps described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, conventional processor, controller, microcontroller, state machine, etc. A processor can also be implemented as a combination of computing devices, e.g., a combination of a DSP and a microprocessor, a plurality of microprocessors, one or more microprocessors in conjunction with a DSP core, or any other such configuration. In addition, the term “processing” is a broad term meant to encompass several meanings including, for example, implementing program code, executing instructions, manipulating signals, filtering, performing arithmetic operations, and the like.

The modules can include, but are not limited to, any of the following: software or hardware components such as software, object-oriented software components, class components and task components, processes, methods, functions, attributes, procedures, subroutines, segments of program code, drivers, firmware, microcode, circuitry, data, databases, data structures, tables, arrays, or variables.

The steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, a DVD, or any other form of storage medium known in the art. A storage medium is coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Although the foregoing invention has been described in terms of certain preferred embodiments, other embodiments will be apparent to those of ordinary skill in the art from the disclosure herein. Additionally, other combinations, omissions, substitutions and modifications will be apparent to the skilled artisan in view of the disclosure herein. It is contemplated that various aspects and features of the invention described can be practiced separately, combined together, or substituted for one another, and that a variety of combinations and subcombinations of the features and aspects can be made and still fall within the scope of the invention. Furthermore, the systems described above need not include all of the modules and functions described in the preferred embodiments. Accordingly, the present invention is not intended to be limited by the reaction of the preferred embodiments, but is to be defined by reference to the appended claims.

Additionally, all publications, patents, and patent applications mentioned in this specification are herein incorporated by reference to the same extent as if each individual publication, patent, or patent application was specifically and individually indicated to be incorporated by reference.

1. (canceled)

2. A method for processing physiological measurements, the method comprising:
receiving, by a medical device management system, physiological measurement data from a portable medical device, wherein the physiological measurement data comprises identification information associated with a user of the portable medical device;
identifying, by the medical device management system, a user account of the medical device management system based on the identification information;
initiating, by the medical device management system, a calibration mode on the portable medical device;
transitioning, by the medical device management system, the portable medical device from a measurement mode to a calibration mode in response to initiation of the calibration mode;
restricting, by the medical device management system, the portable medical device from performing at least one physiological measurement while in calibration mode;
transitioning, by the medical device management system, the portable medical device from the calibration mode to the measurement mode upon completion of calibration;
processing, by the medical device management system, the physiological measurement data to determine at least one physiological parameter associated with the physiological measurement data;
transmitting the determined at least one physiological parameter to the portable medical device for display; and
storing, by the medical device management system, the physiological measurement data and the determined at least one physiological parameter in association with the identified user account.

3. The method of claim 2, further comprising:
accessing, by the medical device management system, a user-specific calibration profile associated with the user account, wherein the user-specific calibration profile is generated from historical invasive and non-invasive physiological measurement data associated with the user account and is stored in a data store of the medical device management system remote from the portable medical device.

4. The method of claim 3, further comprising:
updating, by the medical device management system, the user-specific calibration profile in the data store based on subsequent measurement data received from the portable medical device.

5. The method of claim 3, wherein processing the physiological measurement data to determine at least one physiological parameter comprises using the user-specific calibration profile.

6. The method of claim 2, wherein the portable medical device transitions to a standby mode if calibration is not completed within a predetermined time period.

7. The method of claim 2, wherein restricting the portable medical device from performing at least one physiological measurement comprises disabling non-invasive measurements while permitting invasive measurements during calibration mode.

8. The method of claim 2, wherein the portable medical device displays a calibration status indicator while in calibration mode.

9. The method of claim 3, wherein the user-specific calibration profile is synchronized across a plurality of portable medical devices associated with the user account.

10. The method of claim 2, wherein the physiological measurement data comprises at least one of invasive blood glucose measurement data and non-invasive blood glucose measurement data.

11. The method of claim 2, wherein the medical device management system communicates with the portable medical device over a wireless communication network.

12. The method of claim 2, wherein the physiological measurement data is contained in a structured data file.

13. The method of claim 2, further comprising:
providing, by the medical device management system, the determined at least one physiological parameter to a user account different from the identified user account based on one or more privacy settings of the identified user account.

14. The method of claim 2, wherein the medical device management system is configured to process physiological measurement data associated with at least one additional physiological parameter selected from the group consisting of: Oxygen Saturation (SpO2), Total Hemoglobin (SpHb), Alkaline Phosphatase (SpALP), Total Cholesterol (SpChol), High-Density Lipoprotein (SpHDL), and Total Cholesterol Divided by High Density Lipoprotein (SpChol/SpHDL).

15. The method of claim 2, further comprising:
logging, by the medical device management system, calibration events and measurement results in association with the user account.

16. A medical device management system comprising:
a data store configured to store user account information;
a processor in communication with the data store and configured to:
receive physiological measurement data from a portable medical device, wherein the physiological measurement data comprises identification information associated with a user of the portable medical device;
identify a user account based on the identification information;
initiate a calibration mode on the portable medical device;
transition the portable medical device from a measurement mode to a calibration mode in response to initiation of the calibration mode;
restrict the portable medical device from performing at least one physiological measurement while in calibration mode;
transition the portable medical device from the calibration mode to the measurement mode upon completion of calibration;
process the physiological measurement data to determine at least one physiological parameter associated with the physiological measurement data;
transmit the determined at least one physiological parameter to the portable medical device for display; and
store the physiological measurement data and the determined at least one physiological parameter in association with the identified user account.

17. The system of claim 16, wherein the processor is further configured to:
access a user-specific calibration profile associated with the user account, wherein the user-specific calibration profile is generated from historical invasive and non-invasive physiological measurement data associated with the user account and is stored in the data store remote from the portable medical device.

18. The system of claim 17, wherein the processor is further configured to:
update the user-specific calibration profile in the data store based on subsequent measurement data received from the portable medical device.

19. The system of claim 17, wherein the processor is further configured to:
process the physiological measurement data to determine at least one physiological parameter using the user-specific calibration profile.

20. The system of claim 17, wherein the user-specific calibration profile is synchronized across a plurality of portable medical devices associated with the user account.
